(19) United States
(12) Patent Application Publication
(10) Pub. No.: US 2024/0290457 A1
(43) Pub. Date: Aug. 29, 2024

(54) ARTIFICIAL INTELLIGENCE APPARATUS FOR PREDICTING AN OUTCOME OF A HAIR TRANSPLANT PROCEDURE

(71) Applicant: Linkov Marketing LLC, New York, NY (US)

(72) Inventors: Milos Vukadinovic, Los Angeles, CA (US); Gary Linkov, New York, NY (US); Marko Vuckovic, Long Island City, NY (US)

(21) Appl. No.: 18/174,829

(22) Filed: Feb. 27, 2023

(51) Int. Cl. G16H 20/40; G06T 7/33; G06T 19/20; G06V 40/18

(52) U.S. Cl. CPC G16H 20/40; G06T 7/337; G06T 19/20; G06V 40/193; G06T 2207/20081; G06T 2207/20084; G06T 2207/30201; G06T 2210/22; G06T 2219/2004; G06T 2219/2016

(57) ABSTRACT

An artificial intelligence apparatus for predicting an outcome of a hair transplant procedure comprising a camera to capture digital image data including portrait photos featuring hairlines, a communication interface to receive and transmit digital image data files including portrait photos featuring a hairline, a display to project digital image data including portrait photos featuring a hairline and a processor configured implementing one or more artificial intelligence models.

### FIELD

This application relates to an artificial intelligence apparatus for predicting an outcome of a hair transplant procedure and, more specifically, to an artificial intelligence apparatus implementing neural network-trained models for predicting an outcome of a hair transplant procedure, including an image alignment model and an augmented image generator model.

### BACKGROUND

Hair transplants are an excellent hair restoration option for patients with androgenic alopecia, the most common type of hair loss. Still, many people avoid this procedure because they are uncomfortable discussing the topic with a doctor or are concerned about the quality of the results. Moreover, hair transplant surgeons may not have the means of effectively communicating how a patient will look after a hair transplant procedure. What is needed is a system and method that helps patients better understand what to expect from a hair transplant procedure and helps surgeons to plan better and effectively execute a hair transplant procedure.

### SUMMARY

An artificial intelligence apparatus for predicting an outcome of a hair transplant procedure is claimed. The artificial intelligence apparatus comprises a camera to capture digital image data including portrait photos featuring hairlines, a communication interface to receive and transmit digital image data files including portrait photos featuring a hairline, a display to project digital image data that include portrait photos featuring a hairline, and a processor. The processor configured to confirm quality of a portrait photo featuring a pre-transplant hairline, the portrait photo originating from the camera or the communication interface, preprocess the portrait photo featuring a pre-transplant hairline whose quality has been confirmed, input the portrait photo featuring a pre-transplant hairline that has been preprocessed into an image alignment model, generate a bounding box surrounding a face included within the portrait photo featuring a pre-transplant hairline using the image alignment model, identify a left eye and a right eye within the face of the portrait photo featuring a pre-transplant hairline using the image alignment model, generate an aligned portrait photo featuring a pre-transplant hairline by rotating the portrait photo so that a line connecting the left eye and right eye is horizontal and by cropping the portrait photo to include only the face within the bounding box, input the aligned portrait photo featuring a pre-transplant hairline into an augmented image generator model, generate a portrait photo featuring a post-transplant hairline using the augmented image generator model, the generated portrait photo featuring a post-transplant hairline version of the pre-transplant hairline within the portrait photo featuring a pre-transplant hairline, and project the portrait photo featuring a pre-transplant hairline alongside the generated portrait photo featuring a post-transplant hairline on the display.

### BRIEF DESCRIPTION OF THE DRAWINGS

The drawings described below are for illustrative purposes only and are not necessarily drawn to scale. The drawings are not intended to limit the scope of the disclosure in any way. The same or like reference numbers are used throughout the drawings to refer to the same or like parts wherever possible.

FIG. 1 illustrates an artificial intelligence apparatus for predicting an outcome of a hair transplant procedure according to an embodiment of the present disclosure.

FIG. 2 illustrates an artificial intelligence training server according to an embodiment of the present disclosure.

FIG. 3 illustrates a block diagram of generative adversarial networks according to the present disclosure.

FIGS. 4A-4B are a flow chart of a method implemented by the artificial intelligence apparatus for predicting an outcome of a hair transplant procedure according to an embodiment of the present disclosure.

FIGS. 5A-5B are a flow chart of a method for training a generator model implemented by the artificial intelligence apparatus for predicting an outcome of a hair transplant procedure according to an embodiment of the present disclosure.

### DETAILED DESCRIPTION

To address the above-described problems, an artificial intelligence apparatus for predicting an outcome of a hair transplant procedure is provided. The artificial intelligence apparatus may implement one or more neural network-based models to help provide a prediction of a potential outcome of a hair transplant procedure.

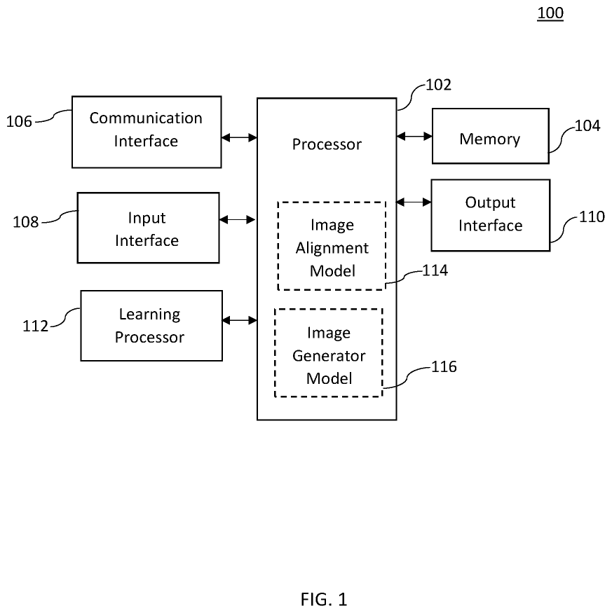

FIG. 1 illustrates an artificial intelligence (AI) apparatus 100 for predicting an outcome of a hair transplant procedure according to an embodiment of the present disclosure. The AI apparatus 100 may be implemented within a stationary apparatus or a mobile apparatus, including a server-based system, a desktop computer, a notebook, a personal digital assistant, a mobile phone, a smartphone, or any other type of appropriate processor-based apparatus known to a person of ordinary skill in the art.

As shown in FIG. 1, the AI apparatus 100 may include a processor 102 in direct communication with at least a memory 104, a communication interface 106, an input interface 108, and an output interface 110. The AI apparatus 100 may further include a learning processor 112 in direct communication with the processor 102.

The memory 104 may be configured to store and retrieve data supporting various functions of the AI apparatus 100, including input data acquired by the input interface 108 and data received by the communication interface 106. Moreover, the memory may store and retrieve AI related data, including learning data, learning models, application programs, learning history, and implemented learned models.

The communication interface 106 may be configured to transmit and receive data to and from external devices, such as other AI apparatuses and AI training servers, using wired or wireless communication technologies. For example, the communication interface 106 may transmit and receive digital image data, user inputs, AI learning models, and control signals to and from external apparatuses and devices.

The communication technology implemented by the communication interface 106 may include GSM (Global System for Mobile communications), CDMA (Code Division Multi Access), LTE (Long Term Evolution), 5G, LAN (Local Area Network), WLAN (Wireless LAN), Wi-Fi (Wireless-Fidelity), Bluetooth™, RFID (Radio Frequency Identification), or any other protocol reasonably known to a person of ordinary skill in the art.

The input interface 108 may be configured to acquire various data types, including being configured as a camera capable of generating and inputting digital image data. Other configurations may include a microphone capable of generating and inputting audio data and a user input interface for inputting control information from a user.

The output interface 110 may be configured to output various data types, including as a display for displaying digital image data. Other configurations may include speakers capable of outputting digital audio data and a haptic module for outputting haptic information.

The processor 102 may be configured to control the components of the AI apparatus 100 to execute determined operations. Specifically, the processor 102 may control at least part of the components of the AI apparatus 100 to implement an application program or one or more learned models stored in the memory 104. When the connection of an external device is required to perform a determined operation, the processor 102 may generate control signals necessary to control the external device and transmit the generated control signals to the external device. Similarly, the processor 102 may acquire and evaluate user input to determine the user's requirements based on the information acquired.

The processor 102 may generate an output from an inputted digital image that includes portrait photos featuring hairlines using at least an AI-based image generator model 116 for generating an augmented version of an inputted portrait photo. In addition, the processor 102 may use an AI-based image alignment model 114 for aligning inputted image data.

The image alignment model and the augmented image generator model may be configured as an artificial neural network learned according to a machine learning algorithm. Moreover, the processor 102 may collect history information, including the operations of the AI apparatus 100 and a user's feedback and may store the collected history information in the memory 104. The collected historical information may be used to update any learned models implemented by the processor 102.

The learning processor 112 may perform AI related processing in conjunction with or as an alternative to the processor 102. The AI related processing performed by the learning processor 112 may include implementing neural network based learned models, including the image alignment model and/or the augmented image generator model. Moreover, the AI related processing performed by the learning processor 112 may include implementing a teaching algorithm for AI-based neural network based models using learning data.

The learning processor 112 may differ from standard central processing units (CPUs), such as the processor 102, in that they include AI-optimized design features that help accelerate the identical, predictable, and independent calculation required by AI algorithms. These design features may include providing for the execution of a large number of calculations in parallel rather than in series, performing calculations at a low precision level, and speeding up memory access. In an exemplary embodiment, the learning processor 112 may be a field programable gate array (FPGA) incorporating large amounts of smaller transistors that run faster and consume less power than the larger transistor often found in standard CPUs.

The AI-based models may be implemented to infer result values for new inputs based on the learning data used to train the models. The inferred result values may then be used to help determine the performance of certain operations by the AI apparatus 100.

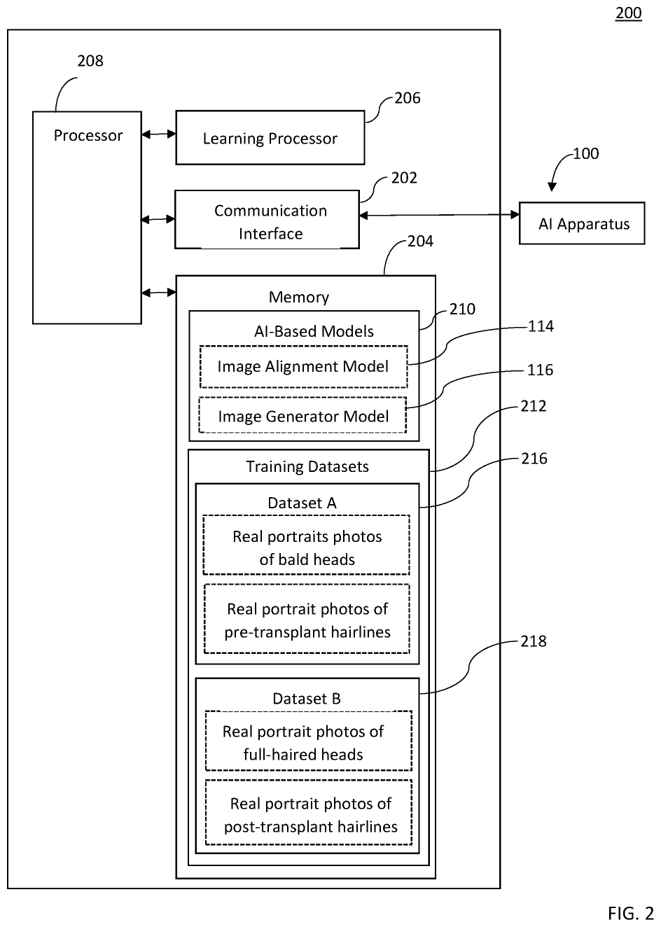

FIG. 2 illustrates an artificial intelligence training server 200 according to an embodiment of the present disclosure. The AI training server 200 may refer to a device or apparatus that implements a machine learning algorithm to train AI-based neural network models.

The AI training server 200 may be comprised of a single local server or a plurality of interconnected servers that provide distributed processing. Moreover, the AI apparatus 100 may be implemented as a component of the AI training server 200, allowing the AI apparatus 100 to perform a portion of the processing initiated, implemented, and controlled by the AI training server 200.

Referring to FIG. 2, the AI training server 200 may include a communications interface 202, a memory 204, a learning processor 206, and a processor 208.

The communication interface 202 may be configured to transmit and receive data to and from external devices such as the AI apparatus 100 and other AI training servers using wire or wireless communication technologies. For example, the communication interface 202 may transmit and receive digital image data, user inputs, AI-based models, and control signals to and from external apparatuses and devices.

As with the communication interface 106 of the AI apparatus 100, the communication technology implemented by the communication interface 202 may include GSM (Global System for Mobile communications), CDMA (Code Division Multi Access), LTE (Long Term Evolution), 5G, LAN (Local Area Network), WLAN (Wireless LAN), Wi-Fi (Wireless-Fidelity), Bluetooth™, RFID (Radio Frequency Identification), or any other reasonable protocol known to a person of ordinary skill in the art.

The memory 204 may be configured to store and retrieve data supporting various functions of the AI training server 200, including AI-based models 210 and training datasets 212 necessary to implement training algorithms.

The stored model may include AI-based models 210 presently learning or previously trained using a training algorithm implemented by the AI training server 200. In the present disclosure, the AI-based models 210 may include at least one of the image alignment model 114 and the image generator model 116.

The training datasets 212 may include data used by a training algorithm, including a training dataset A 216 and a training dataset B 218. The training dataset A 216 may be comprised of digital image data of real portrait photos of bald heads and real portrait photos of pre-transplant hairlines. The training dataset B 218 may be comprised of digital image data of real portrait photos of full-haired heads and real portrait photos of post-transplant hairlines.

As with the AI apparatus 100, the processor 208 may be configured to control the components of the AI training server 200 to execute determined operations. The processor 208 may be further configured to infer result values for new input data using the AI-based models 210 and generate a response or a control command based on these inferred result values.

The processor 208 and the learning processor 206 may, individually or in conjunction, be configured to train artificial intelligence neural networks of the AI-based models 210 within the memory 204 using the learning data in the training datasets 212. While being trained, the AI-based models 210 may be implemented in a state of being mounted on the AI training server 200 or of being mounted on an external device such as the AI apparatus 100.

As with the learning processor 112 of the AI apparatus 100, the learning processor 206 may differ from standard central processing units (CPUs), such as the processor 102, in that they include AI-optimized design features that help accelerate the identical, predictable, and independent calculation required by AI algorithms. In an exemplary embodiment, the learning processor 206 may be a field programable gate array (FPGA) incorporating large amounts of smaller transistors that run faster and consume less power than the larger transistor often found in standard CPUs.

Accordingly, the image alignment model 114 and the image generator model 116 may be trained and executed using any combination of the processor 102 and the learning processor 112 of the AI apparatus 100 and the processor 208 and the learning processor 206 of the AIT training server.

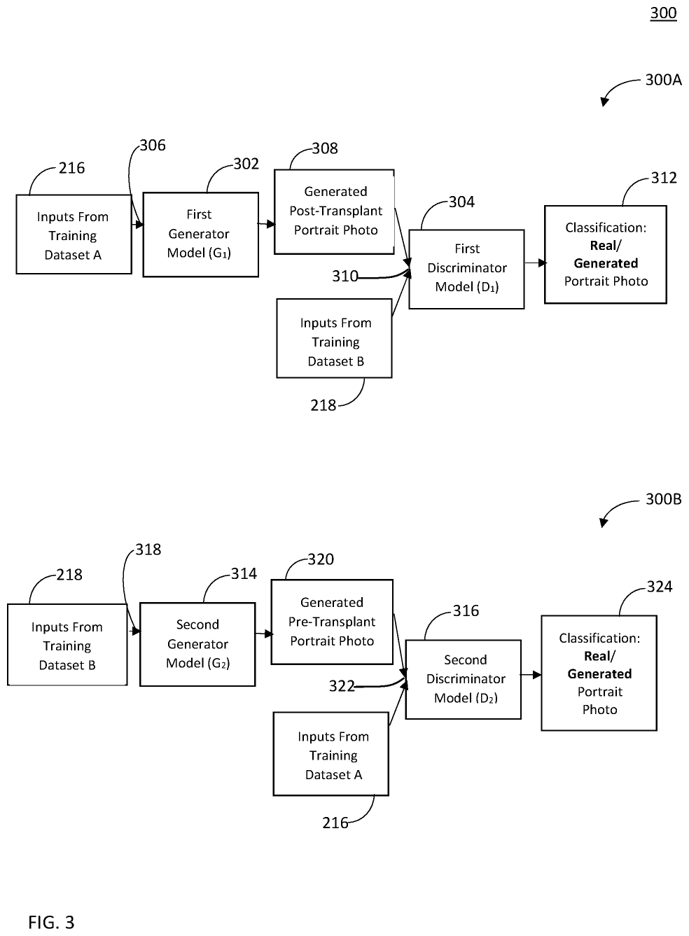

FIG. 3 illustrates a block diagram of machine learning network configurations 300 implemented by the AI training server 200 according to the present disclosure. The machine learning network configurations 300 may include a first machine learning network configuration 300A and a second machine learning network configuration 300B.

According to the present disclosure, the first machine learning network configuration 300A and the second machine learning network configuration 300B may each implement a training algorithm in parallel. A total loss calculation used to achieve conversion of the machine learning network configuration 300 may incorporate loss elements from both the first or second machine learning network configurations 300A, 300B. The loss of one machine learning network configuration being used to help improve the other machine learning network configurations.

In an exemplary embodiment, the first and second machine learning network configurations 300A, 300B may be configured as generative adversarial networks (GAN). Alternatively, the first and second machine learning network configurations 300A, 300B may have any other training network configuration reasonably known to a person of ordinary skill in the art while remaining within the scope of the present disclosure.

As shown in FIG. 3, the first machine learning network configuration 300A may include a first generator model 302 and a first discriminator model 304. Similarly, the second machine learning network configuration 300B may include a second generator model 314 and a second discriminator model 316.

The first generator model 302 may be configured as a convolutional neural network (CNN) outputting probabilities as a tensor variable of similar dimensions as an input tensor variable.

Similarly, the second generator model 314 may be configured as a convolutional neural network (CNN) outputting probabilities as a tensor variable of similar dimensions as an input tensor variable.

The first discriminator model 304 may be configured as a CNN outputting a scalar probability for two possible outcomes based on an inputted tensor variable.

Similarly, the second discriminator model 316 may be configured as a CNN outputting a scalar probability for two possible outcomes based on an inputted tensor variable.

Traditional CNNs have a general configuration that includes stacked convolution layers, each convolutional layer followed by an activation function and a max pooling operation.

A convolutional layer comprises a matrix of filters, each filter applied to a corresponding data input as a dot product resulting in a discreet output value. Applying these filters to the data input enables the neural network to identify specific features and patterns within the inputted data.

The activation function is a non-linear function applied to each discrete output value from the convolutional layer. A typical activation function is a rectified linear unit that converts negative inputs from the convolutional layer to zero while passing on positive inputs. This transformation serves to lighten the downstream computational load.

The max pooling operation progressively reduces the spatial resolution of the neural network between convolutional layers. Specifically, the max pooling operation slides through the input data in strides and outputs the input's maximum value at each stride, dropping the remaining inputs.

In an exemplary embodiment, the first and second generator models 302, 314 may be configured as a residual neural network (RNN) type of CNN. Along with the layers of a traditional CNN, a RNN configuration includes an additional mapping that adds the output from a first layer to a second layer downstream from the first layer, thereby skipping the layers between the first and second layers. As a result, the output of a specific layer may be added to the output of subsequently stacked layers. These additional mappings, called skip connections, help minimize loss and improve the performance of deep neural networks, including CNNs.

Regarding the first machine learning network configuration 300A in FIG. 3, the first generator model 302 receives tensor variable inputs 306 randomly selected from the training dataset A 216 stored in the memory 204 of the AI training server 200. These tensor variable inputs 306 are digital three-dimensional matrix representations of real portrait photos featuring pre-transplant hairlines and bald heads.

Based on the tensor variable inputs 306, the first generator model 302 generates tensor variable outputs 308. These generated tensor variable outputs 308 are digital three-dimensional matrixes representations of portrait photos featuring post-transplant hairlines based on the tensor variable inputs 306.

The first discriminator model 304 receives the generated tensor variable outputs 308 from the first generator model 302 at its input 310. In addition, the first discriminator model 304 also receives at its input 310 a tensor variable randomly selected from the training dataset B 218 stored in the memory 204 of the AI training server 200. These tensor variables from the dataset B are digital three-dimensional matrixes representations of real portrait photos featuring post-transplant hairlines and full-haired heads.

Based on the input 310, the first discriminator model 304 outputs a scalar determination 312 of whether the input 310 was a real digital image from the training dataset B 218, or a generated digital image generated by the first generator model 302. In the present disclosure, the first discriminator model 304 outputs a “1” to designate that an inputted digital image is real and a “0” to designate that an inputted digital image is generated. The first discriminator attempts to distinguish real data from the training dataset B 218 from the data created by the first generator model 302.

Regarding the second machine learning network configuration 300B in FIG. 3, the second generator model 314 receives tensor variable inputs 318 randomly selected from the training dataset B 218. These tensor variable inputs 318 are digital three-dimensional matrix representations of real portrait photos featuring post-transplant hairlines and full-haired heads.

Based on these tensor variable inputs 318, the second generator model 314 generates tensor variable outputs 320. The generated tensor variable outputs 320 are digital three-dimensional matrix representations of portrait photos featuring pre-transplant hairlines.

The second discriminator model 316 receives the generated tensor variable outputs 320 from the output of the second generator model 314 at its input 322. In addition, the second discriminator model 316 also receives at the input 322 a tensor variable randomly selected from the training dataset A 216. These tensor variables randomly selected from the training dataset A 216 being a digital three-dimensional matrix representation of real portrait photos featuring pre-transplant hairlines and bald heads.

Based on the input 322, the second discriminator model 316 outputs a scalar determination 324 of whether the input 322 was a real digital image from the dataset A 216, or a generated digital image generated by the second generator model 314. In the present disclosure, the second discriminator model 316 outputs a “1” to designate that an inputted digital image is real and a “0” to designate that an inputted digital image is generated.

Following each inferred real or fake determination by the first and second discriminator models 304, 316 a total system loss for the first and second machine learning network configurations 300A, 300B across the dataset A 216 and the training dataset B 218 is calculated. The total system loss may be represented as:

$${L}_{Total} = {L}_{G 1} + {L}_{D 1} + {L}_{idl} + {L}_{p ercl} + {L}_{G 2} + {L}_{D 2} + {L}_{i d 2} + {L}_{p e r c 2}$$

The calculated total system loss values are used iteratively to adjust each model's neural network parameters to help minimize subsequent loss values.

The neural network parameters adjusted to minimize loss may include weights, biases, filter types, and any other parameters known to a person of ordinary skill. Each parameter is adjusted by backpropagating a calculated loss through a corresponding CNN.

As an example, within the first and second machine learning network configurations 300A, 300B, the first discriminator loss value (L_{D1}) 316 and the second discriminator loss value (L_{D2}) 332 are each calculated and applied to the first and second discriminator models 304, 316, respectively, to penalize these models when they predict that an inputted digital image is real when it was generated or that an inputted digital image was generated when it is real.

Similarly, the first generator loss values (L_{G1}) and the second generator loss value (L_{G2}) are calculated and applied to the first and second generator models 302, 314, respectively, to penalize these models when the first and second discriminator models 304, 316 correctly determine that a digital image has been generated.

The components of the total system loss within the first machine learning network configuration 300A may include a first generator loss value (L_{G1}), a first discriminator loss value (L_{D1}), and a first style loss value (L_{id1}).

The first generator loss value (L_{G1}) for the first generator model 302 may be represented as:

L_{G1}=MSE(D₁(G₁(X_{A})),1).

A first discriminator loss value (L_{D1}) 316 for the first discriminator model 304 may be represented as:

L_{D1}=0.5(MSE(D₁(X_{B}),1)+MSE(D₁(G₁(X_{A})),0)).

The first style loss value (Liai) for the first generator model 302 may be represented as:

L_{id1}=sum(abs(G₁(X_{A})−X_{A})

The first style loss value (L_{id1}) component functions to help preserve the style of a digital image as originally inputted within a digital image generated by the first generator model 302.

The functions and variables within the first generator loss value (L_{G1}), the first discriminator loss value (L_{D1}) 316, and the first style loss value (L_{id1}) may be defined as:
MSE( ): Mean Square Error
D₁( ): Inferred Output of First Discriminator Model
G₁( ): Inferred Output of First Generator Model
X_{A}: Pre-Transplant Images from Dataset A
X_{B}: Post-Transplant Images from Dataset B
abs( ): Absolute value
sum( ): Summation

The components of the total system loss within the second machine learning network configuration 300B may include a second generator loss value (L_{G2}), a second discriminator loss value (L_{D2}), and a second style loss value (L_{id2}).

The second generator loss value (L_{G2}) for the second generator model 314 may be represented as:

L_{G2}=MSE(D₂(G₂(X_{B})),1).

The second discriminator loss value (L_{D2}) 332 for the second discriminator model 316 may be represented as:

L_{D2}=0.5(MSE(D₂(X_{A}),1)+MSE(D₂(G₂(XB)),0)).

The second style loss value (L_{id2}) for the second generator model 314 may be represented as:

L_{id2}=sum(abs(G₂(X_{B})−X_{B})

The second style loss value (L_{id2}) component functions to help preserve the style of a digital image as originally inputted within a digital image generated by the second generator model 314.

The functions and variables within the second generator loss value (L_{G2}), the second discriminator loss value (L_{D2}) 332, and the second style loss value (L_{id2}) are defined as:
MSE( ): Mean Square Error
D₂( ): Inferred Output of Second Discriminator Model
G₂( ): Inferred Output of Second Generator Model
X_{A}: Pre-Transplant Images from Dataset A
X_{B}: Post-Transplant Images from Dataset B
abs( ): Absolute value
sum( ): Summation

The components of the total system loss which connect the first machine learning network configuration 300A and the second machine learning network configuration 300B may include a first perception loos value (L_{perc1}) and a second perception loss value (L_{perc2}).

The first perception loos value (L_{perc1}) and the second perception loss value (L_{perc2}) component may be represented as:

L_{perc1}=MSE(CNN(G₁(G₂(XB)),CNN(XB))

L_{perc2}=MSE(CNN(G₂(G₁(X_{A})),CNN(X_{A}))

The CNN function within the first perception loss value (L_{perc1}) and the second perception loss value (L_{perc2}) is representative of the output of a convolutional neural network pre-trained to recognize patterns within digital images, including within the first and second machine learning network configurations 300A, 300B.

The first perception loss value (L_{perc1}) and the second perception loss value (L_{perc2}) components within the total system loss (L_{Total}) connect the first and second machine learning network configurations 300A, 300B during training and use the information from one to improve the other.

Specifically, the first perception loss value (L_{perc1}) and the second perception loss value (L_{perc2}) components aim to preserve the features of the original inputted images after cyclically feeding them through both the first and second machine learning network configurations 300A, 300B.

Once convergence across the first and second machine learning network configurations 300A, 300B has been achieved based on the total system loss (L_{Total}), the first generator model 302 may be implemented on the AI apparatus 100 as an image generator model within the AI apparatus 100.

In addition to the total system loss (L_{Total}), convergence may be determined using a Frechet inception distance metric which evaluates how different generated post-surgery digital images are from real post-surgery digital images. Also, a perception test may be implemented by showing test subjects generated post-surgery digital images and real post-surgery digital images and polling the test subjects as to which one has been generated.

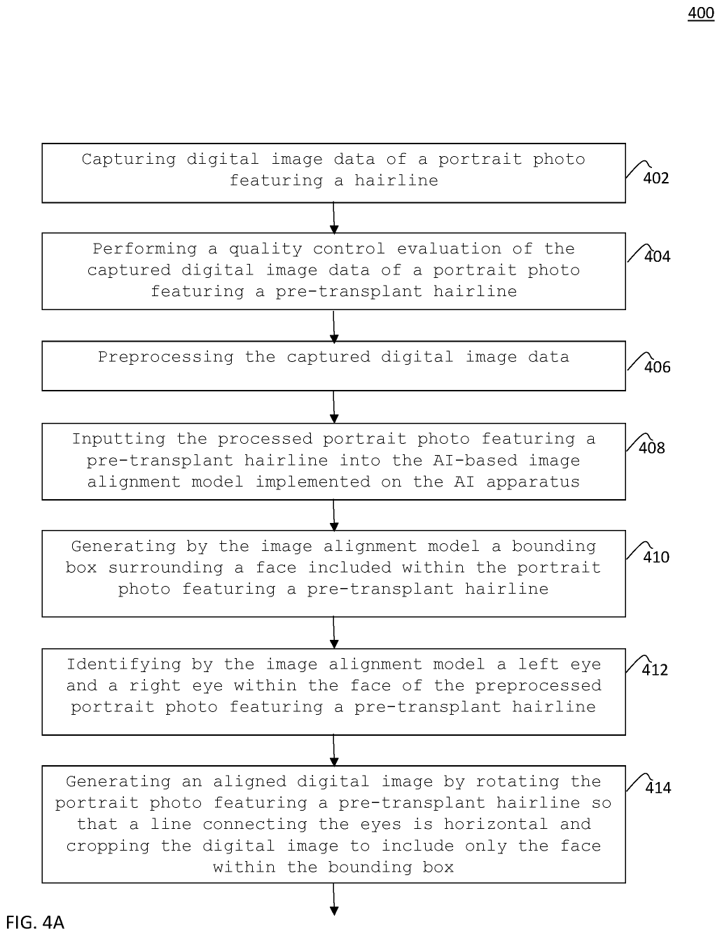

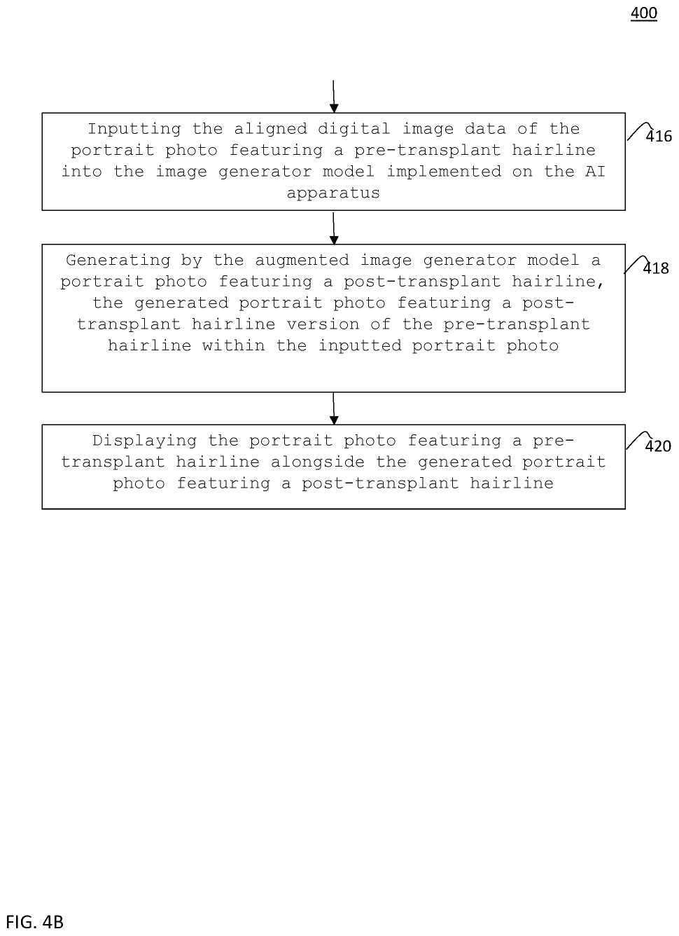

FIGS. 4A-4B are a flow chart of a method 400 implemented by the artificial intelligence apparatus 100 for predicting an outcome of a hair transplant procedure according to an embodiment of the present disclosure.

The method 400 begins in step 402 with the AI apparatus 100 capturing digital image data of a portrait photo featuring a hairline.

The digital image data may be captured using a camera device via the input interface 108. Similarly, the digital image data may be captured using a digital transmission file via the communication interface 106.

Once the digital image data has been captured, the method 400 continues in step 404 with the AI apparatus 100 performing a quality control evaluation of the captured digital image data of a portrait photo featuring a pre-transplant hairline. For example, the portrait photo is evaluated to confirm that a head with a hairline within the photo is present.

Once the quality of the portrait photo featuring a pre-transplant hairline has been verified, the method 400 continues in step 406 with the AI apparatus 100 preprocessing the captured digital image data.

The processing of the digital image data may include resizing and normalizing the digital image data.

Once the digital image data has been preprocessed, the method 400 continues in step 408 with the AI apparatus 100 inputting the processed portrait photo featuring a pre-transplant hairline into the image alignment model 114 implemented on the AI apparatus 100.

Once the digital image data has been inputted, the method 400 continues in step 410 with the image alignment model 114 generating a bounding box surrounding a face included within the portrait photo featuring a pre-transplant hairline.

Next, the method 400 continues in step 412 with the image alignment model 114 identifying a left eye and a right eye within the face of the preprocessed portrait photo featuring a pre-transplant hairline.

Once the bounding box has been generated and the eyes identified, the method 400 continues in step 414 with the AI apparatus 100 generating an aligned digital image by rotating the portrait photo featuring a pre-transplant hairline so that a line connecting the eyes is horizontal and cropping the digital image to include only the face within the bounding box.

Once the aligned digital image has been generated, the method 400 continues in step 416 with the AI apparatus 100 inputting the aligned digital image data of the portrait photo featuring a pre-transplant hairline into the image generator model 116 implemented on the AI apparatus 100.

Once the aligned digital image has been inputted, the method 400 continues in step 418 with the image generator model 116 generating a portrait photo featuring a post-transplant hairline, the generated portrait photo featuring a post-transplant hairline version of the pre-transplant hairline within the inputted portrait photo featuring a pre-transplant hairline.

Lastly, the method 400 ends in step 420 with the AI apparatus displaying the portrait photo featuring a pre-transplant hairline alongside the generated portrait photo featuring a post-transplant hairline.

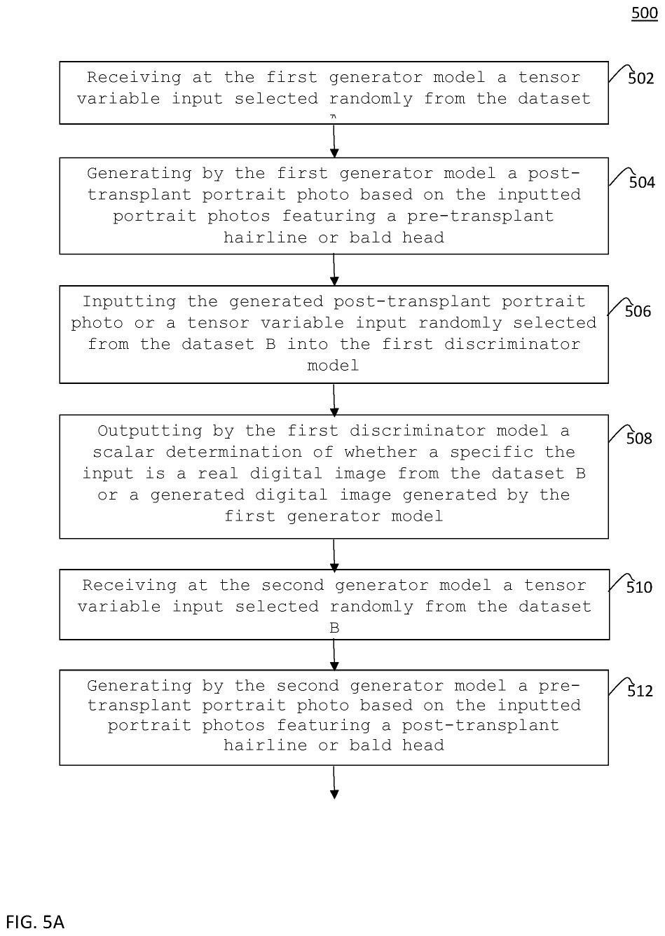

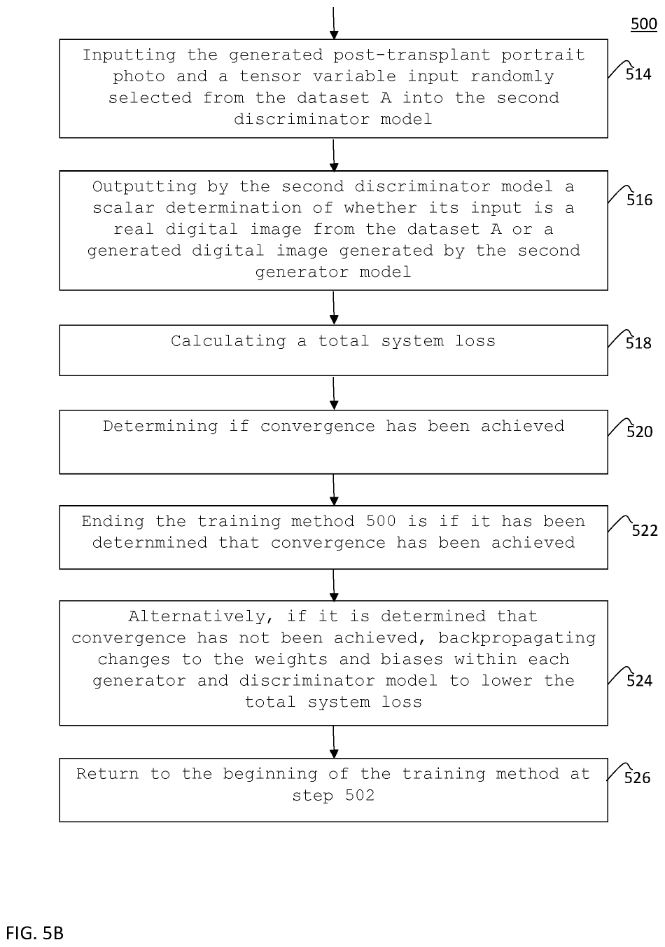

FIGS. 5A-5B are a flow chart of a training method 500 for a generator model implemented by the artificial intelligence apparatus 100 for predicting an outcome of a hair transplant procedure according to an embodiment of the present disclosure.

The training method 500 begins in step 502 with the first generator model 302 receiving a tensor variable input 306 selected randomly from the dataset A 216. The tensor variable input 306 is a digital three-dimensional matrix representation of real portrait photos featuring pre-transplant hairlines and bald heads.

Once a randomly selected tensor variable input 306 from the dataset A 216 has been inputted, the training method 500 continues in step 504 with the first generator model 302 generating a post-transplant portrait photo 308 based on the inputted portrait photos featuring a pre-transplant hairline or bald head.

Once the post-transplant portrait photo 308 has been generated by the first generator model 302, the training method 500 continues in step 506 with providing to the input 310 of the first discriminator model 304 either the generated tensor variable outputs 308 from the first generator 302 or a tensor variable randomly selected from the training dataset B 218. The tensor variable randomly selected from the training dataset B 218 being a digital three-dimensional matrix representation of real portrait photos featuring post-transplant hairlines and full-haired heads.

The training method 500 continues in step 508 with the first discriminator model 304 outputting a scalar determination 312 of whether the input 310 was a real digital image from the training dataset B 218 or a generated digital image generated by the first generator model 302.

Once the first discriminator model has outputted a scalar determination, the training method 500 continues in step 510 with the second generator model 314 receiving a tensor variable input 318 selected randomly from the training dataset B 218. The tensor variable input 318 is a digital three-dimensional matrix representation of real portrait photos featuring post-transplant hairlines and full-haired heads.

Once a randomly selected tensor variable input 318 from the dataset B 218 has been inputted, the training method 500 continues in step 512 with the second generator model 314 generating a pre-transplant portrait photo 320 based on the inputted portrait photos featuring a post-transplant hairline or bald head.

Once the pre-transplant portrait photo 320 has been generated by the second generator model 314, the training method 500 continues in step 514 with providing to the input 322 of the second discriminator model 316 either the generated pre-transplant portrait photo 320 from the second generator model 314 or a tensor variable randomly selected from the dataset A 216. The tensor variable randomly selected from the dataset A 216 being a digital three-dimensional matrix representation of real portrait photos featuring pre-transplant hairlines and bald heads.

The training method 500 continues in step 516 with the second discriminator model 316 outputting a scalar determination 324 of whether the input 322 is a real digital image from the dataset A 216 or a generated digital image generated by the second generator model 314.

Once the second discriminator model 316 outputs a scalar determination, the training method 500 continues in step 518 with the calculation of a total system loss.

Once the total system loss has been calculated, the training method 500 in step 520 determines if convergence has been reached based on the calculated total system loss value.

Once it is determined whether or not convergence has been achieved, the training method 500 ends in step 522 if convergence has been achieved.

Alternatively, if it is determined that convergence has not been achieved, the training method 500 continues in step 524 with backpropagating changes to the weights and biases within each generator and discriminator model to lower the total system loss.

Once changes to the weights and biases have been backpropagated through each generator and discriminator model, the training method 500 in step 526 returns to the beginning of the training method at step 502.

The foregoing description discloses only example embodiments. Modifications of the above-disclosed apparatuses and methods which fall within the scope of this disclosure will be readily apparent to those of ordinary skill in the art.

This disclosure is not intended to limit the invention to the particular apparatuses and/or methods disclosed. Instead, the intention is to cover all modifications, equivalents, and alternatives falling within the scope of the claims.

What is claimed is:

1. An artificial intelligence apparatus for predicting an outcome of a hair transplant procedure comprising:
a camera to capture digital image data including portrait photos featuring hairlines;
a communication interface to receive and transmit digital image data files including portrait photos featuring a hairline;
a display to project digital image data including portrait photos featuring a hairline;
a processor configured to:
confirm quality of a portrait photo featuring a pre-transplant hairline, the portrait photo originating from the camera or the communication interface;
preprocess the portrait photo featuring a pre-transplant hairline whose quality has been confirmed;
generate an aligned portrait photo that has been preprocessed featuring a pre-transplant hairline by rotating the portrait photo so that a line connecting a left eye and a right eye is horizontal and by cropping the portrait photo to include only a face within the portrait photo;
input the aligned portrait photo featuring a pre-transplant hairline into an image generator model; and
generate a portrait photo featuring a post-transplant hairline using the image generator model, the generated portrait photo featuring a post-transplant hairline version of the pre-transplant hairline within the portrait photo featuring a pre-transplant hairline.

2. The artificial intelligence apparatus of claim 1, further comprising the processor configured to:
display the portrait photo featuring a pre-transplant hairline alongside the generated portrait photo featuring a post-transplant hairline on the display.

3. The artificial intelligence apparatus of claim 1, wherein the processor is further configured to transmit the portrait photo featuring a pre-transplant hairline and the generated portrait photo featuring a post-transplant hairline to a predefined surgical clinic via the communication interface.

4. The artificial intelligence apparatus of claim 1, wherein preprocessing of the portrait photo featuring a pre-transplant hairline comprises resizing and normalizing image data of the portrait photo featuring a pre-transplant hairline.

5. The artificial intelligence apparatus of claim 1, further comprising a learning processor configured to implement the image generator model.

6. The artificial intelligence apparatus of claim 1, wherein the image generator model is a convolutional neural network trained to output a portrait photo featuring a post-transplant hairline corresponding to the portrait photo featuring a pre-transplant hairline, the generated portrait photo being substantially indistinguishable from a real digital image of a portrait photo featuring a post-transplant hairline.

7. The artificial intelligence apparatus of claim 6, wherein the image generator model is trained using a generative adversarial network.

8. The artificial intelligence apparatus of claim 7, wherein the generative adversarial network comprises generator models and discriminator models.

9. The artificial intelligence apparatus of claim 8, wherein the generative adversarial network is comprised of a first machine learning network configuration and a second machine learning network configuration;
the first machine learning network configuration inputting a first training dataset into a first generator model, the first training dataset comprised of real portrait photos featuring bald heads and real portrait photos featuring pre-transplant hairlines;
the second machine learning network configuration inputting a second training dataset into a second generator model, the second training dataset comprised of real portrait photos featuring post-transplant hairlines and real portrait photos featuring full-haired heads; and
the first machine learning network configuration and the second machine learning network configuration operating in parallel to train the image generator model.

10. The artificial intelligence apparatus of claim 8, wherein the generator models are residual neural networks, and the discriminator models are patchGAN networks.

11. The artificial intelligence apparatus of claim 7, wherein a convergence criterium for the image generator model is a combination of a Frechet Inception Distance score and a manual visual quality inspection of generated post-transplant portrait photos featuring post transplant hairlines.

12. The artificial intelligence apparatus of claim 11, wherein the Frechet Inception Distance score is determined using generated portrait photos featuring a post-transplant hairline and real portrait photos featuring post-transplant hairlines.

13. An artificial intelligence apparatus for predicting an outcome of a hair transplant procedure comprising:
a camera to capture digital image data including portrait photos featuring hairlines;
a communication interface to receive and transmit digital image data files including portrait photos featuring a hairline;
a display to project digital image data including portrait photos featuring a hairline;
a processor configured to:
confirm quality of a portrait photo featuring a pre-transplant hairline, the portrait photo originating from the camera or the communication interface;
preprocess the portrait photo featuring a pre-transplant hairline whose quality has been confirmed;
input the portrait photo featuring a pre-transplant hairline that has been preprocessed into an image alignment model;
generate a bounding box surrounding a face included within the portrait photo featuring a pre-transplant hairline using the image alignment model;
identify a left eye and a right eye within the face of the portrait photo featuring a pre-transplant hairline using the image alignment model;
generate an aligned portrait photo featuring a pre-transplant hairline by rotating the portrait photo so that a line connecting the left eye and right eye is horizontal and by cropping the portrait photo to include only the face within the bounding box;
input the aligned portrait photo featuring a pre-transplant hairline into an image generator model;
generate a portrait photo featuring a post-transplant hairline using the image generator model, the generated portrait photo featuring a post-transplant hairline version of the pre-transplant hairline within the portrait photo featuring a pre-transplant hairline; and
display the portrait photo featuring a pre-transplant hairline alongside the generated portrait photo featuring a post-transplant hairline on the display.

14. The artificial intelligence apparatus of claim 13, further comprising a learning processor configured to implement the image generator model and the image alignment model.

15. An artificial intelligence method for predicting an outcome of a hair transplant procedure comprising:
providing a camera to capture digital image data including portrait photos featuring hairlines;
providing a communication interface to receive and transmit digital image data files including portrait photos featuring a hairline;
providing a display to project digital image data including portrait photos featuring a hairline;
providing a processor configured to:
confirm quality of a portrait photo featuring a pre-transplant hairline, the portrait photo originating from the camera or the communication interface;
preprocess the portrait photo featuring a pre-transplant hairline whose quality has been confirmed;
generate an aligned portrait photo that has been preprocessed featuring a pre-transplant hairline by rotating the portrait photo so that a line connecting the left eye and right eye is horizontal and by cropping the portrait photo to include only a face within the portrait photo;
input the aligned portrait photo featuring a pre-transplant hairline into an image generator model;
generate a portrait photo featuring a post-transplant hairline using the image generator model, the generated portrait photo featuring a post-transplant hairline version of the pre-transplant hairline within the portrait photo featuring a pre-transplant hairline; and
display the portrait photo featuring a pre-transplant hairline alongside the generated portrait photo featuring a post-transplant hairline on the display.
